SEQUENCE LISTING (1 sequence)

SEQ ID NO: 1 - protein, 573 aa, Artificial Sequence
  Synthesized  H7N9 rH7HA (A/Shanghai/2/2013)
MNTQILVFALIAIIPTNADKICLGHHAVSNGTKVNTLTERGVEVVNATETVERTNIPRICSKGKRTVDLGQCGLLGTITGPPQCDQFLEFSADLIIERREGSDVCYPGKFVNEEALRQILRESGGIDKEAMGFTYSGIRTNGATSACRRSGSSFYAEMKWLLSNTDNAAFPQMTKSYKNTRKSPALIVWGIHHSVSTAEQTKLYGSGNKLVTVGSSNYQQSFVPSPGARPQVNGLSGRIDFHWLMLNPNDTVTFSFNGAFIAPDRASFLRGKSMGIQSGVQVDANCEGDCYHSGGTIISNLPFQNIDSRAVGKCPRYVKQRSLLLATGMKNVPEIPKGRGLFGAIAGFIENGWEGLIDGWYGFRHQNAQGEGTAADYKSTQSAIDQITGKLNRLIEKTNQQFELIDNEFNEVEKQIGNVINWTRDSITEVWSYNAELLVAMENQHTIDLADSEMDKLYERVKRQLRENAEEDGTGCFEIFHKCDDDCMASIRNNTYDHSKYREEAMQNRIQIDPVKLSSGYKDVSGRLVPRGSPMKQIEDKIEEILSKIYHIENEIARIKKLIGEVGHHHHHH

(19) United States
(12) Patent Application Publication
(10) Pub. No.: US 2017/0342113 A1
(43) Pub. Date: Nov. 30, 2017

(54) RECOMBINANT H7 HEMAGGLUTININ AND USE THEREOF

(71) Applicant: National Tsing Hua University, Hsinchu (TW)

(72) Inventors: Suh-Chin WU, Hsinchu (TW); Wen-Chun LIU, Hsinchu (TW); Ting-Hsuan CHEN, Hsinchu (TW)

(21) Appl. No.: 15/255,567

(22) Filed: Sep. 2, 2016

(30) Foreign Application Priority Data
Mar. 15, 2016 (TW) 105107899

(51) Int. Cl. C07K 14/005; C12N 7/00; A61K 39/145; A61K 39/00

(52) U.S. Cl. CPC C07K 14/005; A61K 39/145; C12N 7/00; C12N 2760/16122; C07K 2319/42; C07K 2319/21; A61K 2039/575; A61K 2039/55561; C12N 2760/16134; C12N 2760/16151; C07K 2319/73

(57) ABSTRACT

A recombinant H7 hemagglutinin derived from Chinese hamster ovary (CHO) cell. The recombinant H7 hemagglutinin includes a H7 hemagglutinin domain, a GCN4-pII trimerization motif, and a His-tag. The recombinant H7 hemagglutinin can be prepared as a protective vaccine composition with a pharmaceutically acceptable adjuvant. H7 hemagglutinin specific antibodies are elicited, and protection against H7N9 influenza virus is provided.

### BACKGROUND OF THE INVENTION

### 1. Field of the Invention

The present invention relates to a hemagglutinin from H7N9 influenza virus. More specifically, the present invention discloses a method for preparing recombinant H7 hemagglutinin and use thereof for preparing a vaccine which may elicit specific antibodies.

### 2. Description of the Prior Art

Influenza A viruses have been classified into 17 HA (hemagglutinin, H1-H17) and 10 NA (neuraminidase, N1-N10) serotypes based on their HA and NA protein antigenic characteristics. H7N9 has been described as the result of a H7 reassortant from domestic duck H7N3 viruses, a N9 reassortant from wild bird H11N9 viruses, and six other viral genes from two groups of chicken H9N2 viruses. While they are known to trigger severe pneumonia and/or acute respiratory distress syndrome (ARDS) in humans, avian H7N9 viruses only result in asymptomatic or mild diseases in bird species, which explains their membership in the category of low-pathogenic avian influenza viruses.

Results from molecular analyses indicate that most H7N9 human isolates are characterized by (a) an absence of polybasic amino acids at the HA1/HA2 cleavage site, (b) a HA Q226L mutation, (c) a deletion of 5 amino acids in the NA stalk, and (d) an E627K substitution at PB2 (Dortmans, J. C. et al., 2013; Shi, Y. et al., 2013; Wang, Y. et al., 2013). These and other results underscore the urgent need to develop an effective H7N9 vaccine to reduce the potential for an avian influenza pandemic.

Conventional influenza virus vaccine is prepared by egg-based virus vaccine production, and this preparation method requires expensive 2+ or 3 biosafety level facility. To date, inactivated H7N9 vaccines prepared from reverse-engineered H7N9/PR8 viruses and formulated in oil-in-water emulsions have been shown to induce potent neutralizing antibodies and protective immunity in mice and ferrets (Duan, Y. et al., Response of Mice and Ferrets to a Monovalent Influenza A (H7N9) Split Vaccine. Plos One 2014, 9(6): e99322.; Wu, C. Y. et al., Squalene-adjuvanted H7N9 virus vaccine induces robust humoral immune response against H7N9 and H7N7 viruses. Vaccine 2014.). However, the products via conventional or reverse-engineered method have not been post-translational modified; for instance, disulfide bond formation and complex type glycosylation which facilitate protein folding and stability (Hanson S. R. et al., The core trisaccharide of an N-linked glycoprotein intrinsically accelerates folding and enhances stability. Proc Natl Acad Sci USA 2009, 106(9): 3131-3136.).

### SUMMARY OF THE INVENTION

The present invention provides a recombinant H7 hemagglutinin which does not require expensive experimental devices or facilities to be obtained; nevertheless, post-translational modification (e.g. complex type glycosylation) can still be achieved. The recombinant H7 hemagglutinin is capable of being utilized as a vaccine composition against H7N9 virus.

In this invention, a novel H7N9 influenza subunit vaccine was design as a soluble recombinant H7HA protein which is composed of the ecto-domain of H7 hemagglutinin from the WHO recommend H7N9 vaccine virus strain, A/Shanghai/2/2013 strain, and GCN4pII trimerization motif at the C-terminal of the recombinant protein. With a pharmaceutical acceptable adjuvant, the recombinant H7HA protein may be prepared as a vaccine against H7N9 virus.

First, we design an expression gene for a Chinese hamster ovary (CHO) cell to express rH7HA (CHO-rH7HA cell) and construct a CHO-rH7HA expression plasmid. Then dhFr-(dihydrofolate reductase (DHFR) deficient) gene amplification technology was used to develop high-producing stable CHO cell line. The CHO-rH7HA expression plasmid was further transfected into CHO/dhFr-cell.

Mice were immunized by CHO-rH7HA with various adjuvants at different dosages. Mice sera samples analysis showed that two-dose intramuscular immunizations of CHO-rH7HA elicited rH7HA-specific IgG, IgG1, IgG2a antibodies, showing that CHO-rH7HA immunization elicited rH7HA-specific B cell response, Th1 cells and Th2 cells cellular response. Also, immunization of CHO-rH7HA elicited HI antibody against rH7HA protein and neutralizing antibody against H7N9 virus in sera samples. CHO-rH7HA formulated with PELC/CpG adjuvant induced the highest antibody titers among other formulations with other adjuvants. These data pointed out that the novel CHO-rH7HA vaccine can be produced as an effective H7N9 vaccine for pharmaceutical biologic agent production. Live H7N9 virus challenge experiment results showed that intramuscular immunization with 20 μg CHO-rH7HA formulated with the PELC/CpG adjuvant provided 100% protection against live H7N9 virus.

These and other objectives of the present invention will become obvious to those of ordinary skill in the art after reading the following detailed description of preferred embodiments. It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings:

FIG. 1A illustrates the construction of an expression gene for a Chinese hamster ovary (CHO) cell to express recombinant H7 hemagglutinin protein (rH7HA);

FIG. 1B illustrates that the expression gene in FIG. 1A was cloned into an IKID expression cassette plasmid;

FIG. 2A is a SDS-PAGE qualitative analysis result showing the molecular mass of rH7HA produced by CHO cell line;

FIG. 2B is a Western blotting analysis result showing the molecular weight changes of CHO-rH7HA after Endo H and PNGase F treatment;

FIG. 2C is a gel filtration analysis result showing the protein composition of CHO-rH7HA;

FIG. 3 illustrates glycan composition of CHO-rH7HA;

FIG. 4A illustrates that mice were immunized with 0.2 μg or 2 μg CHO-rH7HA formulated with different adjuvants in week 0 and week 3 and their sera were collected in week 5;

FIG. 4B-4D illustrate antibody responses in mice elicited by CHO-rH7HA vaccines with different adjuvants formulations via intramuscular injection and intranasal immunization;

FIG. 5A-5F illustrate IgG1 and IgG2a subclasses antibody titers elicited by immunization of CHO-rH7HA with different adjuvants formulations;

FIG. 6A-6C illustrate that through immunization of CHO-rH7HA with different adjuvants formulations at different dosages, hemagglutinin inhibition antibody against rH7HA was able to be elicited;

FIG. 6D-6F illustrate that through immunization of CHO-rH7HA with different adjuvants formulations at different dosages, neutralizing antibody titer against H7N9 virus was able to be elicited;

FIG. 7A illustrates protective immunity in the aspect of survival rate against H7N9 virus challenge in mice immunized with CHO-rH7HA plus PELC/CpG adjuvant; and

FIG. 7B illustrates protective immunity in the aspect of body weight loss against H7N9 virus challenge in mice immunized with CHO-rH7HA plus PELC/CpG adjuvant.

### DESCRIPTION OF THE PREFERRED EMBODIMENTS

Reference will now be made in detail to the preferred embodiments of the present invention, examples or explanations of which are illustrated in the accompanying drawings. Wherever possible, the same reference numbers are used in the drawings and the description to refer to the same or like parts.

1. Preparation of Recombinant H7 Hemagglutinin Protein (rH7HA)

a. Design an Expression Gene for a Chinese Hamster Ovary (CHO) Cell to Express rH7HA (CHO-rH7HA Cell) and Construct a CHO-rH7HA Expression Plasmid

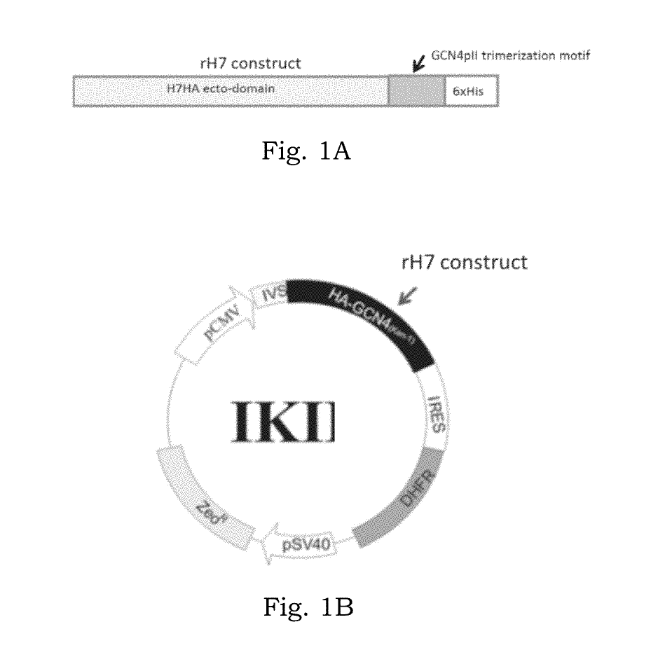

Refer to FIG. 1A. Term definition: the CHO-rH7HA cell represents the Chinese hamster ovary cell which is capable of stably expressing recombinant H7 hemagglutinin protein. The expression gene of CHO-rH7HA cell was constructed by utilizing hemagglutinin cDNA sequences of A/Shanghai/2/2013 (H7N9) virus strain. Transmembrane and cytoplasmic domains at the C terminus of full-length hemagglutinin were deleted and replaced with a leucine zipper GCN4-pII sequence (MKQIEDKIEEILSKIYHIENEIARIKKLIGEV) for trimerization in front of a thrombin cleavage site, ending with a His-tag to facilitate purification. Refer to FIG. 1B. The expression gene was cloned into an IKID expression cassette plasmid containing a pCMV promoter, IVS, IRES-driven DHFR and pSV40 driven Zeocin-resistant gene.

b. Transfection and Single Cell Cloning

To obtain CHO-rH7HA cells, CHO/dhFr-(dihydrofolate reductase (DHFR) deficient) cells were transfected into the plasmid mentioned above, and underwent Zeocin selection. CHO/dhFr-cell line named ATCC CRL-9096 was obtained from Bioresource Collection and Research Center in Taiwan. CHO/dhFr-cell lacked DHFR and could not synthesize ribonucleosides (RNS) and deoxyribonucleosides (dRNS). TurbofFect Transfection reagent (Thermo Scientific) was used to perform DNA transfection into CHO/dhFr-cell. Under nonselective conditions, CHO/dhFr-cells were maintained in Minimum Essential Medium Alpha medium (MEM-α) with ribonucleosides (RNS) and deoxyribonucleosides (dRNS) (Invitrogen), supplemented with 10% fetal bovine serum. 48 hours after transfection, medium was replaced with MEM-α without RNS and dRNS supplemented with 10% dialyzed fetal bovine serum (DF) (Invitrogen) and 200 μg/ml Zeocin (Invitrogen).

After 2 weeks of selection with Zeocin, remaining cells which stably carried the CHO-rH7HA expression plasmid were collected and diluted to 1 cell/100 μl for single colony culture in each well of 96-well plates. After 1 week of incubation at 37° C., wells containing only single cell colony were confirmed by visual inspection under microscopy, and single cell colony in each of those wells was transfer to 24-well plates, incubated for 3 days for cell amplification. To select the CHO-rH7HA cells and eliminate those that were not, the medium sample from each well was collected, and analyzed by Western blotting with anti-rH7HA antibody. CHO-rH7HA cell clones were selected for further steps to obtain high rH7HA producing CHO cell clones.

c. Obtain High rH7HA Producing CHO Cell Clones by Dhfr Gene Amplification and Purification of CHO-rH7HA

To increase the yield of CHO-rH7HA, each clone mentioned above underwent dhfr (DHFR) gene amplification to amplify rH7HA gene copy number. DHFR conversed folate to tetrahydrofolate which participated in the synthesis of GMP and AMP from purine, dTMP from dUMP, and glycine from serine, so dhFr deficient cells must be cultured in medium supplied with RNS and dRNS. Medium of each clone was replaced with MEM-α supplemented with 10% DF (Invitrogen) without RNS or dRNS, so the dhfr gene in the CHO-rH7HA expression plasmid became an essential gene that kept the cells alive. At the presence of MTX (methotrexate), DHFR inhibitor, dhfr gene in the CHO-rH7HA expression plasmid must be amplified and inserted into cell chromosome to develop MTX-resistance cell for cell survival. To obtain CHO cell clones with high rH7HA gene copy number, MTX was added to each cell clone and the concentration of MTX was stepwise increased (0.02 μM, 0.08 μM, 0.32 μM, 1 μM). Cell clones that survived from 1 μM MTX treatment was collected and analyzed by Western blotting with anti-rH7HA antibody to confirm CHO-rH7HA expression. Cell clones which were eventually selected were named 1B1 and further cultured for CHO-rH7HA production. CHO-rH7HA was purified using nickel-chelated affinity chromatography (Tosoh), dialyzed with PBS and stored at −20° C. In the embodiment, the CHO-rH7HA has the following amino acid sequence (SEQ ID NO:1).

2. Analysis of CHO-rH7HA

a. SDS-PAGE

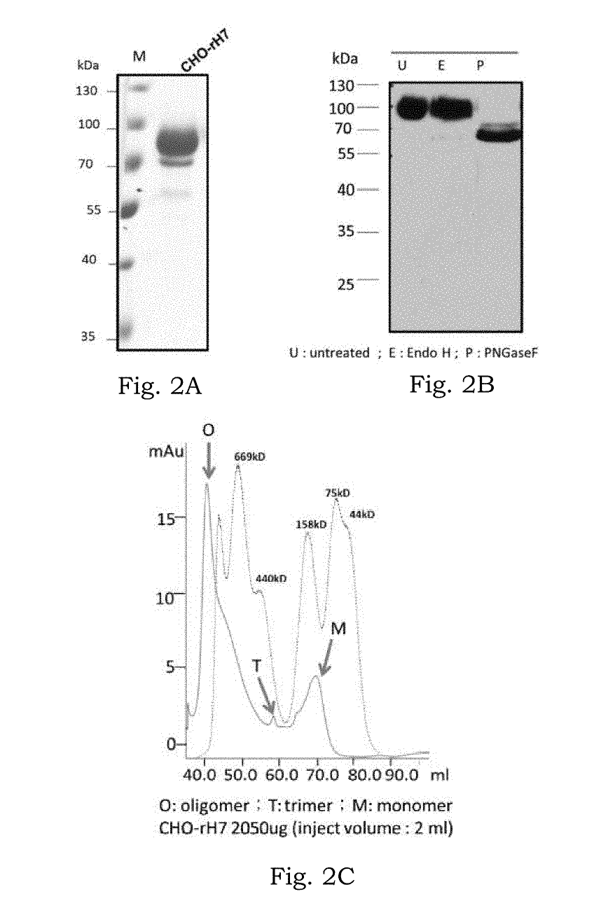

Tris-glycine SDS-polyacrylamide Gel Electrophoresis (SDS-PAGE) was used to analyze proteins expression. 5% stacking gel (3.4 ml H2O with 830 μl 30% acrylamide mix, 630 μl 1M Tris (pH 6.8), 50 μl 10% SDS, 50 μl 10% ammonium persulfate and 5 μl TEMED) was loaded on 12% separating gel (3.3 ml H2O with 4 ml 30% acrylamide mix, 2.5 ml 1M Tris (pH 8.8), 100 μl 10% SDS, 100 μl 10% ammonium persulfate and 10 μl TEMED). The sample ran under 150V for 2 hours. After electrophoresis, the SDS-PAGE gel was stained with 0.25% Coomassie Brilliant Blue R-250 (Sigma) overnight. Then, to de-stain the gel, destained buffer (300 ml methanol, 100 ml acetic acid and 600 ml ddH2O) was used. Refer to FIG. 2A. Via SDS-PAGE to perform qualitative analysis, the molecular mass of CHO-rH7HA is about 100 kDa.

b. Western Blotting

To confirm the characterization of N-linked glycans of CHO-rH7HA, Endo H was used to cleave mannose-terminated N-Glycans; PNGase F was used to cleave all N-linked glycans. 1˜2 μg proteins were mixed with 5 μl loading dye containing DTT and heated in boiling water for 5 mins. CHO-rH7HA were mixed with denaturing buffer in 3:1 ratio and boiled for 10 min. Then the samples were treated with Endo H (NEW ENGLAND BioLabs) in which 1 μg boiled proteins were mixed with 1 μl 10× denature buffer for 10 min, and then double-distilled water was added so that the total volume would be 10 μl. 2 μl 10×G5 buffer, 1.5 μl Endo H, 6.5 μl double-distilled water were further added to the mixture (total volume 20 μl), and the mixture was incubated at 37° C. for 2 hours. The samples were also treated with PNGaseF (NEW ENGLAND BioLabs) in which 1 μg boiled proteins were mixed with 1 μl 10× denature buffer for 10 min, and then double-distilled water was added so that the total volume would be 10 μl. 2 μl 10×G7 buffer, 2 μl 10% NP40 buffer, 1.5 μl PNGase F, 4.5 μl double-distilled water were further added to the mixture (total volume 20 μl), and the mixture was incubated at 37° C. for 2 hours. Tris-glycine SDS-polyacrylamide Gel Electrophoresis (SDS-PAGE) was used to analyze proteins expression. The sample ran under 150V for 2 hours. After electrophoresis, the gel was transferred onto a nitro-cellulose (NC) paper under 135V; the transferring process proceeded approximately 35 mins. 5% milk was used to block the NC paper for 2 hours or overnight. Afterwards, anti-His conjugated HRP antibody (GeneTex) was added in 1:5,000 dilutions with TBST buffer, and waited for 1 hour. A substrate was then used as a detection reagent. Refer to FIG. 2B. After PNGase F treatment, the molecular weights of CHO-rH7HA were decreased.

c. Gel Filtration Chromatography

1 mg of proteins were analyzed by HiLoad 16/60 superdex 200 pg gel column (GE-Healthcare) pre-equilibrated with 0.005M Tris buffer with 0.1M NaCl (pH=8), and the eluted proteins were monitored at 280 nm by Akta prime plus system (GE-Healthcare). To identify the molecular weights of the protein samples, protein molecular samples from GE-Healthcare were used to generate standard curves in advance. Refer to FIG. 2C. Gel filtration analysis showed that CHO-rH7HA was majorly composed of oligomer form protein with minor trimer and monomer form protein.

d. Glycan Analysis

Purified CHO-rH7HA were analyzed for glycan structures according to the method of Royle (Royle et al., Detailed structural analysis of N-glycans released from glycoproteins in SDS-PAGE gel bands using HPLC combined with exoglycosidase array digestions. Methods in molecular biology 2006, 347: 125-143.). Samples were analyzed by SDS-PAGE (Criterion TGX, Biorad) and the SDS-PAGE gels were stained with Coomassie blue. Gel bands were cut to 1 mm³ pieces, frozen at −20° C. overnight, washed with acetonitrile and 20 mM sodium bicarbonate (1:1) and dried in a SpeedVac centrifuge. Glycans were removed from the protein samples by using PNGase F (Promega) at 37° C. overnight. The glycans were removed from the gel by using sonication in water, desalted by Dowex, and filtered through a 45 μm filter. The glycans were dried down in the SpeedVac centrifuge and labelled with 2-aminobenzamide (2-AB). After removing excess 2-AB label, HILIC-HPLC (X-Bridge amide 3.5 μm column) was used to separate the samples to obtain glycan structures. The 2-AB labelled glycans were digested with Jack Bean α-mannosidase (Prozyme) then the HILIC-HPLC was used to confirm the glycan structures once again. A 2-AB labelled dextran ladder standard was also separated by the HILIC-HPLC and used to generate a 5^{th} order polynomial to provide glucose unit (GU) values for the individual peaks which would recognize glycans in protein samples. GU values were compared to those available in the NIBRT GlycoBase database. Refer to FIG. 3. Glycan profile analysis showed that CHO-rH7HA contains majorly complex type N-linked glycans.

3. Immunization Assay of CHO-rH7HA

a. Preparation of PELC/CpG Adjuvant

In this invention, PELC/CpG is a pharmaceutical acceptable adjuvant which was improved based on PELC developed by Dr. Huang, Ming His from Taiwan National Health Research Institutes (Huang et al., Formulation and Immunological Evaluation of Novel Vaccine Delivery Systems Based on Bioresorbable Poly(ethylene glycol)-block-poly(lactide-co-ε-caprolactone). Wiley InterScience 2009, 90B: 832-841.). The PELC/CpG adjuvant was formulated by combining 10% PELC and 10 μg CpG oligodeoxynucleotide in PBS.

PELC is a water-in-oil-in-water emulsion adjuvant in which the composition is similar to MF59 developed by Novartis. The main difference between PELC and MF59 is that the hydrophilic emulsifier in PELC was ameliorated from biodegradable polymer poly(ethylene glycol)-block-poly(lactide-co-ε-caprolactone (PEG-b-PLACL) approved of being utilized in human body by FDA to replace poisonous Tween 80. The hydrophilic part of PELC is water-soluble polyethylene glycol (PEG) and the hydrophobic part of PELC is biodegradable polylactic acid caprolactone (PLC). The composition of PELC comprises squalene and emulsifier (bioabsorbable polymer/hydrophobic excipient Span 85), and the manufacturing process of PELC comprises emulsion and dispersing.

The hydrophilic feature of emulsifier can be controlled by the molecular mass of hydrophilic and hydrophobic compositions in the emulsifier. As the emulsifier enters an organism, the emulsifier would be hydrolyzed into lactic acid and other byproducts which can be converted via Krebs cycle into harmless CO₂ and H₂O and discharged with PEG. In accordance with the information stated above, The PELC/CpG adjuvant is considered to be safe for it can be catabolized.

b. Mouse Immunization

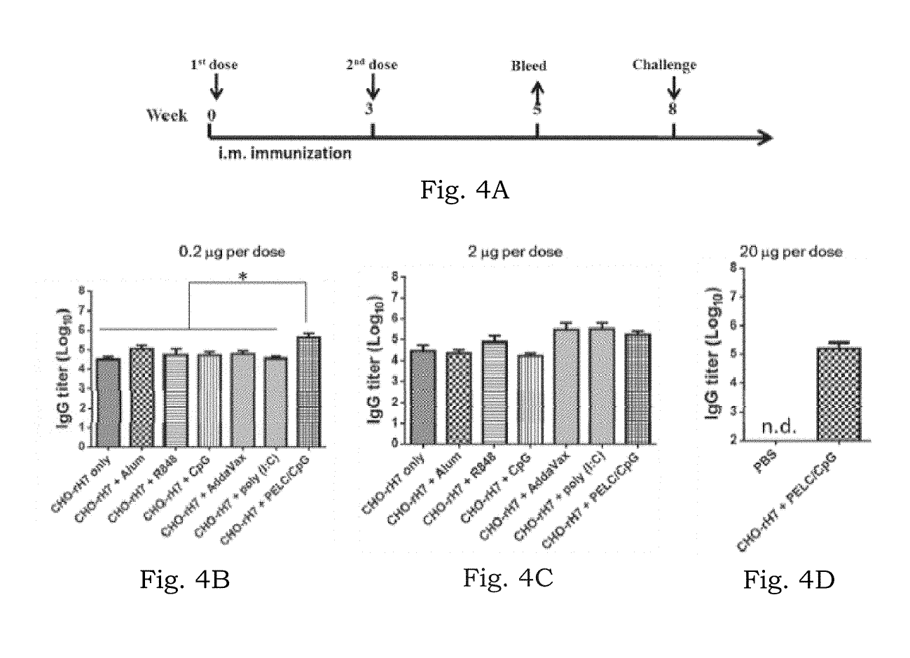

There were two ways to immunize a mouse in this invention, intramuscular injection and intranasal immunization. Refer to FIG. 4A. Through intramuscular immunization regimen, mice were immunized with 0.2 μg or 2 μg CHO-rH7HA formulated with different adjuvants in week 0 and week 3 and their sera were collected in week 5. Refer to FIG. 4B-4D. 6 to 8 weeks BALB/c mice were purchased from Taiwan National Laboratory Animal Center. Five mice in each group were immunized twice via intramuscular injection with different vaccine formulations dissolved in 200 μl PBS, comprising PBS, 0.2 μg and 2 μg CHO-rH7HA without adjuvant or with 300 μg alum adjuvant, 10 μg R848, 10 μg CpG, 50% AddaVax, 10 μg poly (I:C) and mixture consisted of 10% PELC with 10 μg CpG (PELC/CpG). Blood samples were collected at 14 days after the second dose of immunization. Different vaccine formulations were prepared in PBS (30 μl total volume per mouse) for intranasal immunization, comprising PBS, 10 μg CHO-rH7HA without adjuvant and 10 μg CHO-rH7HA formulated with PELC/CpG. Mice were anesthetized with 30 mg/kg Zoletil 50 (Virbac) via intraperitoneal injection prior to each immunization. Afterwards, 15 μl of prepared vaccines were dropped into each nostril three times over a three-week interval. Serum samples were collected 2 weeks after the third immunization. Serum samples were inactivated at 56° C. for 30 minutes and stored at −20° C. for the following assays. As shown in FIG. 4B-4D, mice were immunized and rH7HA-specific IgG antibody titers were elicited by CHO-rH7HA vaccines with different adjuvants formulations.

c. Titer of rH7HA-Specific IgG

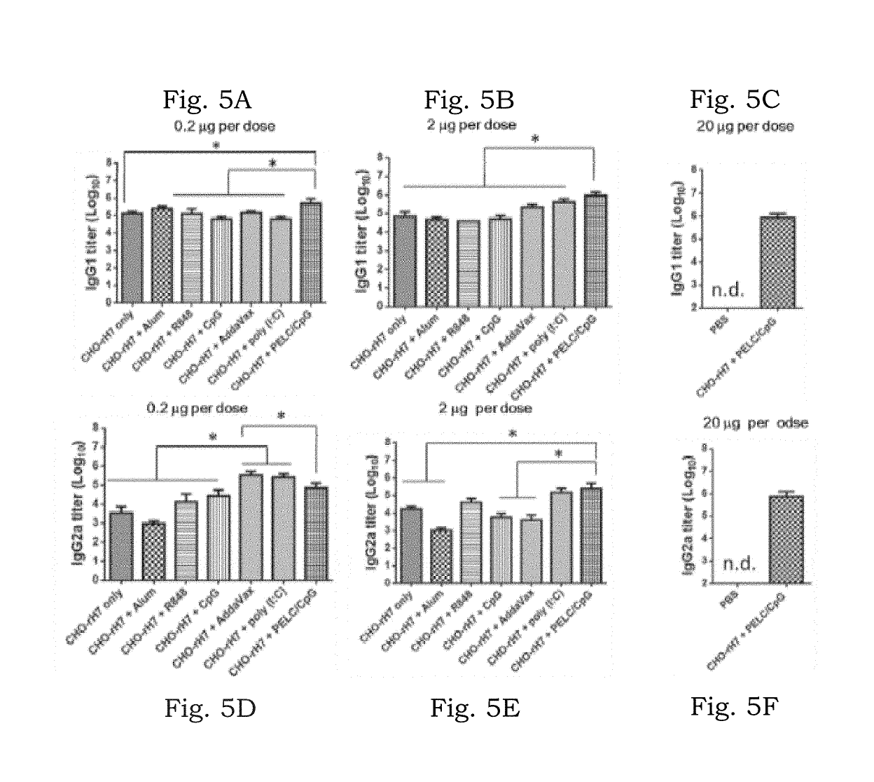

2 μg/ml of purified CHO-rH7HA were coated on 96-well plates overnight and then blocked with ELISA blocking buffer (PBS and 1% BSA) for 1 hour. Afterwards, each well was incubated with two-fold serial diluted sera samples for 1 hour and then subsequently washed by PBST (PBS and 0.05% Tween-20). Samples were incubated for 1 hour with anti-mouse IgG conjugated HRP (1:30000), anti-mouse IgG1 conjugated HRP (1:50000) or anti-mouse IgG2a conjugated HRP (1:50000). Then, plates were further washed by PBST twice. Finally, samples were incubated with TMB substrate in the dark for 15 minutes, and then added ELISA stop solution (2N H₂SO₄). The value of OD₄₅₀ nm was measured by a spectrophotometer. Immunized with CHO-rH7HA, rH7HA-specific IgG was elicited in mice sera samples shown in FIG. 4B-4D; rH7HA-specific IgG1 was elicited in mice sera samples shown in FIG. 5A-5C; rH7HA-specific IgG2a was elicited in mice sera samples shown in FIG. 5D-5F. The results implied that under the immunization regimen with 0.2 μg, 2 μg and 20 μg dosage of CHO-rH7HA plus various adjuvants formulations, rH7HA-specific B cell, Th1 cell and Th2 cell immune responses were able to be elicited.

d. Hemagglutinin Inhibition Assay

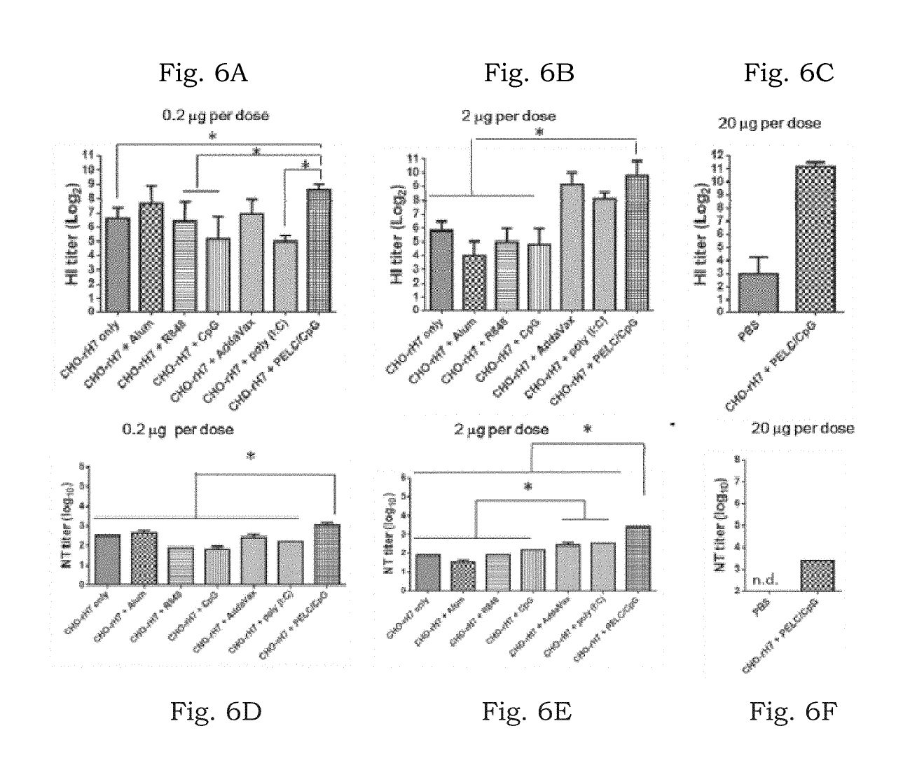

Serum samples were treated with receptor-destroying enzyme (Denka Seiken) overnight at 37° C., then incubated 30 minutes at 56° C. Samples were serial-diluted two-fold (starting from 1:10) and incubated with 4 HA units of CHO-rH7HA for 30 minutes at room temperature. Turkey RBCs (0.5%) were then added to the treated serum samples and held for 30 minutes at room temperature. HI titers were determined as the reciprocal of the highest dilution in which hemagglutination was completely inhibited. As shown in FIG. 6A-6C, under the immunization regimen with CHO-rH7HA plus various adjuvants formulations, rH7HA-specific HI antibody was able to be elicited in the serum samples.

e. Neutralization Assay

MDCK cells (1.5×104/well) were cultured overnight in 96-well microtiter plates. Serum samples were two-fold serial-diluted, co-incubated with equal volumes of H7N9 virus diluent (A/Taiwan/01/2013; 100 TCID50/well) for 1 hour at 4° C., then added to the prepared MDCK cells and incubated for 4 days at 37° C. for virus replication. Infectivity was determined as the presence of cytopathic effect observed on day 4. Neutralizing titers were defined as the reciprocals of the highest serum dilutions in which H7N9 virus infectivity was neutralized in 50% of wells compared to uninfected cells. As shown in FIG. 6D-6F, under the immunization regimen with CHO-rH7HA plus various adjuvants formulations, neutralizing antibody against H7N9 virus was able to be elicited in the serum samples.

Refer to FIGS. 4B-4D, 5A-5F and 6A-6F, CHO-rH7HA is capable of being a basis for preparing a vaccine, and in order to elicit maximum antibody titer, the PELC/CpG adjuvant appears to be the most suitable pharmaceutical acceptable adjuvant compared to other kinds of adjuvant. In accordance with Figures and animal research data presented and stated above, CHO-rH7HA quips the potential for becoming a biological agent; moreover, CHO-rH7HA with the PELC/CpG adjuvant is able to be prepared as a vaccine for protection against H7N9 virus.

f. Virus Challenges

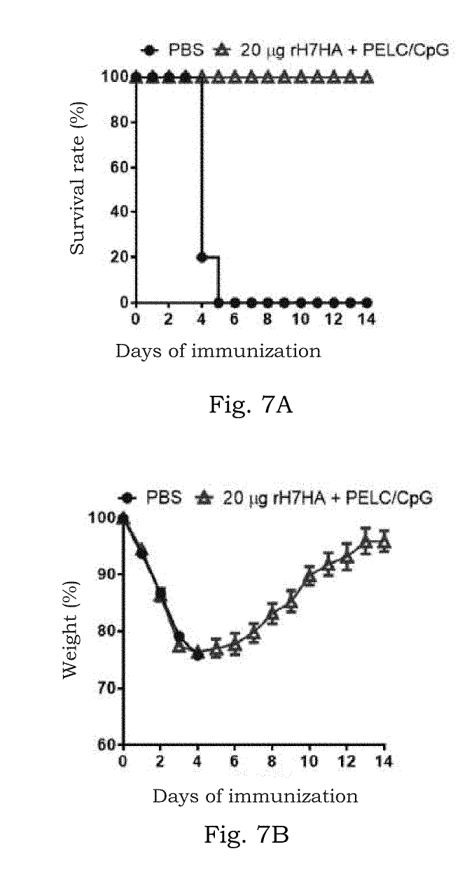

Refer to FIG. 7A-7B. Three weeks after the final immunizations, mice were anesthetized and intranasally challenged with 10 LD50 of the H7N9 virus (A/Taiwan/01/2013) at a volume of 50 μl. PBS-immunized mice were used as a mock control. Mouse survival and weight loss were monitored daily for 14 days. Body weight loss >25% was used as an end-point. As shown in FIG. 7A, under the immunization with CHO-rH7HA plus the PELC/CpG adjuvant, mice had gained full immune protection against H7N9 virus.

Based on the embodiments and Figures described and presented above, inoculation of CHO-rH7HA with the PELC/CpG adjuvant may elicit rH7HA-specific IgG, HI and neutralizing antibodies against H7N9 virus, in which the CHO-rH7HA along with the PELC/CpG adjuvant have the potential for preparing an effective vaccine against H7N9 virus. Besides, the CHO cell clones can be utilized as a mass production method for rH7HA which is a basic and essential biological agent material.

g. Statistical Analyses

All results were analyzed using one-way ANOVAs and Tukey's tests (GraphPad Prism v5.03), with p<0.05 indicating statistical significance. All experiments were performed at least two times each.

It will be apparent to those skilled in the art that various modifications and variations can be made to the present invention without departing from the scope or spirit of the invention. In view of the foregoing, it is intended that the present invention cover modifications and variations of this invention provided they fall within the scope of the invention and its equivalent.

What is claimed is:

1. A recombinant H7 hemagglutinin comprising:
a H7 hemagglutinin domain, the H7 hemagglutinin domain is derived from ecto-domain of H7 hemagglutinin from WHO recommend H7N9 vaccine virus strain, A/Shanghai/2/2013 strain;
a GCN4-pII trimerization motif, the GCN4-pII trimerization motif comprises a leucine zipper GCN4-pII sequence (MKQIEDKIEEILSKIYHIENEIARIKKLIGEV); and
a His-tag;
wherein the recombinant H7 hemagglutinin is produced by a Chinese hamster ovary (CHO) cell carrying a plasmid expressing the recombinant H7 hemagglutinin, and the recombinant H7 hemagglutinin comprises an amino acid sequence as SEQ ID NO: 1.

2. The recombinant H7 hemagglutinin of claim 1, wherein the Chinese hamster ovary (CHO) cell is made to be dihydrofolate reductase (DHFR) deficient.

3. The recombinant H7 hemagglutinin of claim 1, wherein the recombinant H7 hemagglutinin contains complex type N-linked glycans.

4. The recombinant H7 hemagglutinin of claim 1, wherein the recombinant H7 hemagglutinin comprises oligomer, trimer and monomer.

5. The recombinant H7 hemagglutinin of claim 1, wherein the recombinant H7 hemagglutinin elicits specific IgG antibody, IgG1 antibody and IgG2a antibody.

6. The recombinant H7 hemagglutinin of claim 1, wherein the recombinant H7 hemagglutinin elicits specific hemagglutinin inhibition (HI) antibody.

7. The recombinant H7 hemagglutinin of claim 1, wherein the recombinant H7 hemagglutinin elicits neutralizing antibody against H7N9 virus.

8. A method for preparing a recombinant H7 hemagglutinin, comprising:
(1) designing a gene expressing the recombinant H7 hemagglutinin, further comprising:
using a hemagglutinin cDNA sequence of A/Shanghai/2/2013 (H7N9) virus strain as a template;
constructing a leucine zipper GCN4-pII trimerization motif, transmembrane and cytoplasmic domains at the C terminus of the hemagglutinin cDNA sequence are deleted and replaced with a leucine zipper GCN4-pII sequence (MKQIEDKIEEILSKIYHIENEIARIKKLIGEV) for trimerization in front of a thrombin cleavage site; and
adding a His-tag, the His-tag is added at an end of the GCN4-pII trimerization motif;
(2) constructing a plasmid expressing the recombinant H7 hemagglutinin, further comprising:
cloning the gene into an IKID expression cassette plasmid comprising a pCMV promoter, an IVS gene, an IRES-driven DHFR gene and a pSV40 driven Zeocin-resistant gene; and
amplifying the IRES-driven DHFR gene;
(3) transfecting the plasmid into a Chinese hamster ovary (CHO) cell;
(4) the Chinese hamster ovary (CHO) cell carrying the plasmid is undergone single cell cloning with Zeocin selection; and
(5) improving production of the recombinant H7 hemagglutinin, further comprising:
adding MTX (methotrexate) for the Chinese hamster ovary (CHO) cell carrying the plasmid to become MTX-resistant.

9. The method for preparing the recombinant H7 hemagglutinin of claim 8, wherein the Chinese hamster ovary (CHO) cell is made to be dihydrofolate reductase (DHFR) deficient.

10. The method for preparing the recombinant H7 hemagglutinin of claim 8, wherein the recombinant H7 hemagglutinin contains complex type N-linked glycans.

11. The method for preparing the recombinant H7 hemagglutinin of claim 8, wherein the recombinant H7 hemagglutinin comprises oligomer, trimer and monomer.

12. The method for preparing the recombinant H7 hemagglutinin of claim 8, wherein the recombinant H7 hemagglutinin elicits specific IgG antibody, IgG1 antibody and IgG2a antibody.

13. The method for preparing the recombinant H7 hemagglutinin of claim 8, wherein the recombinant H7 hemagglutinin elicits specific hemagglutinin inhibition (HI) antibody.

14. The method for preparing the recombinant H7 hemagglutinin of claim 8, wherein the recombinant H7 hemagglutinin elicits neutralizing antibody against H7N9 virus.

15. A recombinant H7 hemagglutinin vaccine and a pharmaceutical acceptable adjuvant, wherein the recombinant H7 hemagglutinin vaccine comprises the recombinant H7 hemagglutinin of claim 1.

16. The recombinant H7 hemagglutinin vaccine and a pharmaceutical acceptable adjuvant of claim 15, wherein the pharmaceutical acceptable adjuvant comprises a PELC/CpG adjuvant.

17. The recombinant H7 hemagglutinin vaccine and a pharmaceutical acceptable adjuvant of claim 15, wherein the recombinant H7 hemagglutinin vaccine elicits specific IgG antibody, IgG antibody and IgG2a antibody.

18. The recombinant H7 hemagglutinin vaccine and a pharmaceutical acceptable adjuvant of claim 15, wherein the recombinant H7 hemagglutinin vaccine elicits specific hemagglutinin inhibition (HI) antibody.

19. The recombinant H7 hemagglutinin vaccine and a pharmaceutical acceptable adjuvant of claim 15, wherein the recombinant H7 hemagglutinin vaccine elicits neutralizing antibody against H7N9 virus.
